SEQUENCE LISTING (34 sequences)

SEQ ID NO: 1 - DNA, 82 bp, synthetic construct
  source 1..82: mol_type = other DNA
  misc_feature 1: note = phosphate
  misc_binding 1..20: bound_moiety = Probe binding region on target sequence of crtM gene of Lactiplantibacillus sp.
  misc_structure 21..40: note = Nucleotides constituting the stem in the stem-loop structure
  misc_structure 41..62: note = stem-loop structure (T7 promoter complementary sequence, loop sequence and T7 promoter sequence)
  misc_structure 63..82: note = Nucleotides constituting the stem in the stem-loop structure
acgataaagcaaagtggcggccctatagtgagtcgtattaatttcgcgacaacacgcgaaattaatacgactcactataggg

SEQ ID NO: 2 - DNA, 62 bp, synthetic construct
  source 1..62: mol_type = other DNA
  misc_feature 1..38: note = Aptamer sequence
  misc_binding 39..62: bound_moiety = Probe binding region on target sequence of crtM gene of Lactiplantibacillus sp.
ggatccattcgttacctggctctcgccagtcgggatccttcacgtgcgattagcaataattc

SEQ ID NO: 3 - DNA, 82 bp, synthetic construct
  source 1..82: mol_type = other DNA
  misc_feature 1: note = phosphate
  misc_binding 1..20: bound_moiety = Probe binding region on target sequence of crtN gene of Lactiplantibacillus sp.
  misc_structure 21..40: note = Nucleotides constituting the stem in the stem-loop structure
  misc_structure 41..62: note = stem-loop structure (T7 promoter complementary sequence, loop sequence and T7 promoter sequence)
  misc_structure 63..82: note = Nucleotides constituting the stem in the stem-loop structure
agtcactgtggatcggatgaccctatagtgagtcgtattaatttcgcgacaacacgcgaaattaatacgactcactataggg

SEQ ID NO: 4 - DNA, 63 bp, synthetic construct
  source 1..63: mol_type = other DNA
  misc_feature 1..38: note = Aptamer sequence
  misc_binding 39..63: bound_moiety = Probe binding region on target sequence of crtN gene of Lactiplantibacillus sp.
ggatccattcgttacctggctctcgccagtcgggatccggaaaatcagcattacataggacat

SEQ ID NO: 5 - DNA, 83 bp, synthetic construct
  source 1..83: mol_type = other DNA
  misc_feature 1: note = phosphate
  misc_binding 1..21: bound_moiety = Probe binding region on target sequence of crtM gene of Enterococcus sp.
  misc_structure 22..41: note = Nucleotides constituting the stem in the stem-loop structure
  misc_structure 42..63: note = stem-loop structure (T7 promoter complementary sequence, loop sequence and T7 promoter sequence)
  misc_structure 64..83: note = Nucleotides constituting the stem in the stem-loop structure
tggcttcgatcaacaatgggtccctatagtgagtcgtattaatttcgcgacaacacgcgaaattaatacgactcactataggg

SEQ ID NO: 6 - DNA, 62 bp, synthetic construct
  source 1..62: mol_type = other DNA
  misc_feature 1..38: note = Aptamer sequence
  misc_binding 39..62: bound_moiety = Probe binding region on target sequence of crtM gene of Enterococcus sp.
ggatccattcgttacctggctctcgccagtcgggatccgcatttcttccaagcgttttttca

SEQ ID NO: 7 - DNA, 83 bp, synthetic construct
  source 1..83: mol_type = other DNA
  misc_feature 1: note = phosphate
  misc_binding 1..21: bound_moiety = Probe binding region on target sequence of crtN gene of E. gilvus
  misc_structure 22..41: note = Nucleotides constituting the stem in the stem-loop structure
  misc_structure 42..63: note = stem-loop structure (T7 promoter complementary sequence, loop sequence and T7 promoter sequence)
  misc_structure 64..83: note = Nucleotides constituting the stem in the stem-loop structure
aaagcagctcgatcatgggaaccctatagtgagtcgtattaatttcgcgacaacacgcgaaattaatacgactcactataggg

SEQ ID NO: 8 - DNA, 64 bp, synthetic construct
  source 1..64: mol_type = other DNA
  misc_feature 1..38: note = Aptamer sequence
  misc_binding 39..64: bound_moiety = Probe binding region on target sequence of crtN gene of E. gilvus
ggatccattcgttacctggctctcgccagtcgggatcctccacctttcataaaatgaatgccat

SEQ ID NO: 9 - DNA, 83 bp, synthetic construct
  source 1..83: mol_type = other DNA
  misc_feature 1: note = phosphate
  misc_binding 1..21: bound_moiety = Probe binding region on target sequence of crtM gene of L. citreum
  misc_structure 22..41: note = Nucleotides constituting the stem in the stem-loop structure
  misc_structure 42..63: note = stem-loop structure (T7 promoter complementary sequence, loop sequence and T7 promoter sequence)
  misc_structure 64..83: note = Nucleotides constituting the stem in the stem-loop structure
gccaactattgaccaacgcatccctatagtgagtcgtattaatttcgcgacaacacgcgaaattaatacgactcactataggg

SEQ ID NO: 10 - DNA, 60 bp, synthetic construct
  source 1..60: mol_type = other DNA
  misc_feature 1..38: note = Aptamer sequence
  misc_binding 39..60: bound_moiety = Probe binding region on target sequence of crtM gene of L. citreum
ggatccattcgttacctggctctcgccagtcgggatcctcgtgagcaatcattgcagtct

SEQ ID NO: 11 - DNA, 83 bp, synthetic construct
  source 1..83: mol_type = other DNA
  misc_feature 1: note = phosphate
  misc_binding 1..21: bound_moiety = Probe binding region on target sequence of crtN gene of L. mesenteroides
  misc_structure 22..41: note = Nucleotides constituting the stem in the stem-loop structure
  misc_structure 42..63: note = stem-loop structure (T7 promoter complementary sequence, loop sequence and T7 promoter sequence)
  misc_structure 64..83: note = Nucleotides constituting the stem in the stem-loop structure
ttcgcaactacggcatcacaaccctatagtgagtcgtattaatttcgcgacaacacgcgaaattaatacgactcactataggg

SEQ ID NO: 12 - DNA, 64 bp, synthetic construct
  source 1..64: mol_type = other DNA
  misc_feature 1..38: note = Aptamer sequence
  misc_binding 39..64: bound_moiety = Probe binding region on target sequence of crtN gene of L. mesenteroides
ggatccattcgttacctggctctcgccagtcgggatccttgtcattgtgtaaggaaaatcagcg

SEQ ID NO: 13 - DNA, 20 bp, synthetic construct
  source 1..20: mol_type = other DNA
  misc_binding 1..20: bound_moiety = Complementary sequence of target sequence of crtM gene of Lactiplantibacillus sp.
acgataaagcaaagtggcgg

SEQ ID NO: 14 - DNA, 24 bp, synthetic construct
  source 1..24: mol_type = other DNA
  misc_binding 1..24: bound_moiety = Complementary sequence of target sequence of crtM gene of Lactiplantibacillus sp.
ttcacgtgcgattagcaataattc

SEQ ID NO: 15 - DNA, 25 bp, synthetic construct
  source 1..25: mol_type = other DNA
  misc_binding 1..25: bound_moiety = Complementary sequence of target sequence of crtN gene of Lactiplantibacillus sp.
ggaaaatcagcattacataggacat

SEQ ID NO: 16 - DNA, 25 bp, synthetic construct
  source 1..25: mol_type = other DNA
  misc_binding 1..25: bound_moiety = Complementary sequence of target sequence of crtN gene of Lactiplantibacillus sp.
ggaaaatcagcattacataggacat

SEQ ID NO: 17 - DNA, 21 bp, synthetic construct
  source 1..21: mol_type = other DNA
  misc_binding 1..21: bound_moiety = Complementary sequence of target sequence of crtM gene of Enterococcus sp.
tggcttcgatcaacaatgggt

SEQ ID NO: 18 - DNA, 24 bp, synthetic construct
  source 1..24: mol_type = other DNA
  misc_binding 1..24: bound_moiety = Complementary sequence of target sequence of crtM gene of Enterococcus sp.
gcatttcttccaagcgttttttca

SEQ ID NO: 19 - DNA, 21 bp, synthetic construct
  source 1..21: mol_type = other DNA
  misc_binding 1..21: bound_moiety = Complementary sequence of target sequence of crtN gene of E. gilvus
aaagcagctcgatcatgggaa

SEQ ID NO: 20 - DNA, 26 bp, synthetic construct
  source 1..26: mol_type = other DNA
  misc_binding 1..26: bound_moiety = Complementary sequence of target sequence of crtN gene of E. gilvus
tccacctttcataaaatgaatgccat

SEQ ID NO: 21 - DNA, 21 bp, synthetic construct
  source 1..21: mol_type = other DNA
  misc_binding 1..21: bound_moiety = Complementary sequence of target sequence of crtM gene of L. citreum
gccaactattgaccaacgcat

SEQ ID NO: 22 - DNA, 22 bp, synthetic construct
  source 1..22: mol_type = other DNA
  misc_binding 1..22: bound_moiety = Complementary sequence of target sequence of crtM gene of L. citreum
tcgtgagcaatcattgcagtct

SEQ ID NO: 23 - DNA, 21 bp, synthetic construct
  source 1..21: mol_type = other DNA
  misc_binding 1..21: bound_moiety = Complementary sequence of target sequence of crtN gene of L. mesenteroides
ttcgcaactacggcatcacaa

SEQ ID NO: 24 - DNA, 26 bp, synthetic construct
  source 1..26: mol_type = other DNA
  misc_binding 1..26: bound_moiety = Complementary sequence of target sequence of crtN gene of L. mesenteroides
ttgtcattgtgtaaggaaaatcagcg

SEQ ID NO: 25 - DNA, 25 bp, synthetic construct
  source 1..25: mol_type = other DNA
atgaagcaagtatcgattattggcg

SEQ ID NO: 26 - DNA, 21 bp, synthetic construct
  source 1..21: mol_type = other DNA
ttaagcctccttaagggctag

SEQ ID NO: 27 - DNA, 25 bp, synthetic construct
  source 1..25: mol_type = other DNA
atgaagaaggtaatagtaataggtg

SEQ ID NO: 28 - DNA, 20 bp, synthetic construct
  source 1..20: mol_type = other DNA
tcgcggtaaacaaaagttgg

SEQ ID NO: 29 - DNA, 24 bp, synthetic construct
  source 1..24: mol_type = other DNA
atgcagaacatttcaatagtaggc

SEQ ID NO: 30 - DNA, 24 bp, synthetic construct
  source 1..24: mol_type = other DNA
ttatgccacaatatatttcaccgc

SEQ ID NO: 31 - DNA, 25 bp, synthetic construct
  source 1..25: mol_type = other DNA
atgaaaacaatctcaataattggag

SEQ ID NO: 32 - DNA, 23 bp, synthetic construct
  source 1..23: mol_type = other DNA
tcacttaatgtgttttgaaaagg

SEQ ID NO: 33 - DNA, 20 bp, synthetic construct
  source 1..20: mol_type = other DNA
agagtttgatcmtggctcag

SEQ ID NO: 34 - DNA, 20 bp, synthetic construct
  source 1..20: mol_type = other DNA
ggttaccttgttacgacttc

(19) United States
(12) Patent Application Publication
(10) Pub. No.: US 2024/0376553 A1
(43) Pub. Date: Nov. 14, 2024

(54) PROBE SET FOR ISOTHERMAL ONE-POT REACTION FOR DETECTING STRAINS WITH BIOLOGICALLY ACTIVE BIOSYNTHETIC PATHWAY AND USES THEREOF

(71) Applicant: POSTECH Research and Business Development Foundation, Pohang-si (KR)

(72) Inventors: Jeong Wook LEE, Pohang-si (KR); Mi Bang KIM, Pohang-si (KR); Ye Rim SHIN, Pohang-si (KR); Chang Ha WOO, Pohang-si (KR)

(73) Assignee: POSTECH Research and Business Development Foundation, Pohang-si (KR)

(21) Appl. No.: 18/658,036

(22) Filed: May 8, 2024

(30) Foreign Application Priority Data
May 8, 2023 (KR) 10-2023-0059393
Apr. 26, 2024 (KR) 10-2024-0056315

(51) Int. Cl. C12Q 1/689; C12Q 1/682

(52) U.S. Cl. CPC C12Q 1/689; C12Q 1/682; C12Q 2600/156; C12Q 2600/16

(57) ABSTRACT

The present disclosure relates to a probe set for isothermal one-pot reaction for detecting strains with biologically active biosynthetic pathway and uses thereof. The platform according to the present disclosure selectively binds only to the functional metabolite essential biosynthetic gene cluster by using the probe set according to the present disclosure, and thus it is possible to perform phenotype-based screening on strains isolated using the probe set according to the present disclosure, thereby effectively screening strains with biologically biosynthetic pathway (functional metabolite biosynthetic strains) in a fast and accurate manner. Furthermore, the platform according to the present disclosure is able to utilize RNA as well as DNA as targets, thereby detecting gene clusters that are actually actively expressed.

### CROSS-REFERENCE TO RELATED APPLICATIONS

This application claims priority under 35 U.S.C. § 119 to Korean Patent Application No. 10-2023-0059393, filed on May 8, 2023 and Korean Patent Application No. 10-2024-0056315, filed on Apr. 26, 2024 in the Korean Intellectual Property Office, the disclosure of which is incorporated herein by reference in its entirety.

### INCORPORATION BY REFERENCE OF SEQUENCE LISTING

The content of the electronically submitted sequence listing, file name: Q298241_sequence listing as filed. XML; size: 54,949 bytes; and date of creation: May 8, 2024, filed herewith, is incorporated herein by reference in its entirety.

### TECHNICAL FIELD

The following disclosure relates to a probe set for isothermal one-pot reaction for detecting strains with biologically active biosynthetic pathway and uses thereof.

### BACKGROUND

The discovery of functional microorganisms is the most fundamental research step not only for the study of microbial metabolic functions, but also for the development of functional materials using useful genetic resources and the development of technologies for the commercialization of useful biomaterials. However, biosynthetic cluster that confers activity to functional microorganisms may exhibit different sequences depending on the microbial species, even for the same active substance, and even if a specific cluster is detected, it is not possible to confirm whether the pathway containing this sequence is active. Therefore, the present disclosure aims to present a new platform capable of being used for novel functional microorganisms with various target useful substances through detection of active pathways using specific DNA probes.

Biologically active pathways have been reported in a variety of microorganisms to date. Some strains of the genus Bacillus produce 1-deoxynojirimycin (DNJ), which has antidiabetic activity, and the gabT1, yktc1, and gutB1 gene clusters are essential for the production of DNJ. In addition, some Chromobacterium violaceum strains produce violacein, which has antibacterial, antiparasitic, and anticancer activity, and the vioA, vioB, vioE, vioD, and vioC gene clusters are essential for its production. In addition, staurosporine having anticancer activity is produced by Streptomyces staurosporeus and requires the staP, staG, staN, staMA, and staMB gene clusters, and Rebeccamycin exhibiting antitumor properties is produced by Lentzea aerocolonigenes and requires the rebH, rebo, rebD, rebC, rebP, rebG, and rebM gene clusters.

In addition to this, the detection of active pathways containing essential biosynthetic gene clusters is essential for the production of various active substances. The present disclosure was conducted by selecting C30 carotenoid produced by lactic acid bacteria as a representative example of these active substances.

Carotenoids are isoprenoid compounds with various functions including acting as precursors to vitamin A, removal of reactive oxygen species (ROS), and exhibiting antioxidant and anticancer activities. Approximately 750 carotenoids have been isolated from nature and are already applied in various industries. However, the industrial application of previously studied carotenoids has been faced with side effects, by-product generation, and low production yields, and thus in recent years, researchers have endeavored to overcome these limitations by developing a new class of safe carotenoids.

In addition, when carotenoid production is induced by applying metabolic engineering techniques based on genetic research to lactic acid bacteria, it is difficult to maintain a balance with existing metabolic pathways, and in particular, the storage capacity of the host must be considered for highly lipophilic compounds such as carotenoids, and due to the nature of carotenoids located in the cell membrane of microorganisms, induction of high levels of carotenoid production may cause cell death. Thus, the production of carotenoids from modified lactic acid bacteria is challenging. Therefore, in order to obtain high yields of carotenoids resources, from natural carotenoid-producing microorganisms have been studied in recent years.

There are many different types of carotenoids produced by bacteria, which may be categorized by the length of carbon backbone thereof. The C50 carotenoids, decaprenoxanthin and bacterioruberin have been reported to be produced by strains of the genus Arthrobacter and the genus Haloferax, respectively, and the C40 carotenoids, cantaxanthin, astaxanthin, and lutein have been reported to be produced by strains of the genus Rhodococcus and the genus Gordonia. It has also been reported that the C30 carotenoids, 4,4′-diaponeurosporene and staphyloxanthin are typically produced by strains of the genus Lactiplantibacillus and Staphylococcus aureus, respectively.

As in the previous example, the gene clusters for biosynthesis are essential for detecting active pathways, and the biosynthetic gene clusters for each carotenoid are as follows. The essential genes for the biosynthesis of bacterioruberin, a C50 carotenoid, are crtB, crtI, lyeJ, and cruF, while the essential genes for the biosynthesis of lycopene, a C40 carotenoid, are crtB and crtI. In addition, it has been reported that the essential genes for biosynthesis of 4,4′-diaponeurosporene, a C30 carotenoid, are crtM and crtN, and the essential genes for biosynthesis of staphyloxanthin are crtM, crtN, crtP, crtQ, and crtO. Among them, the study of C30 carotenoids derived from lactic acid bacteria was first reported in 2010. This study found the lactic acid bacteria-derived pigment as 4,4′-diaponeurosporene, a C30 carotenoid, and reported its biosynthetic pathway. In addition, recent studies have reported colitis amelioration, immune-boosting effects, and antioxidant activity of 4,4′-diaponeurosporene produced by lactic acid bacteria.

However, since 4,4′-diaponeurosporene is biosynthesized by a limited number of lactic acid bacteria and is relatively new to the field, the isolation of the microorganisms responsible for its biosynthesis remains relatively poorly understood. In addition, 4,4′-diaponeurosporene is biosynthesized from FPP by CrtN (dehydrosqualene desaturase) and CrtM (dehydrosqualene synthase), but since these biosynthetic genes are strain-specific, the number of lactic acid bacteria that biosynthesize 4,4′-diaponeurosporene is limited, even if they belong to the same species, and a complete genetic analysis of each strain is necessary to confirm the production of the carotenoid from new, previously unidentified strains.

Genome mining techniques, which target DNA to detect microbial resources, have been used to date primarily to isolate useful microorganisms, but are challenged by the time-consuming of steps between isolating microorganisms from their source and confirming their useful activity and the complexity of the steps requiring different primers and PCR conditions for different DNA sequences. Identification of lactic acid bacteria isolated from different sources is difficult, especially for lactic acid bacteria with high homology of 16S rRNA sequences.

Further, the sample pretreatment process that is necessary to isolate microorganisms from a source may lead to a decrease in isolation efficiency in the discovery of diverse microorganisms from multiple sources. Therefore, it is necessary to apply effective methods to obtain strains that biosynthesize functional metabolite with various useful activities.

### SUMMARY

An embodiment of the present disclosure is directed to providing a probe set for isothermal one-pot reaction for detecting strains with biologically active biosynthetic pathway (hereinafter referred to as functional metabolite biosynthetic strains) and uses thereof.

Specifically, the present disclosure aims to provide a composition for detecting functional metabolite biosynthetic strains comprising: a probe set for isothermal one-pot reaction for detecting a target nucleic acid including a first probe and a second probe.

In addition, another embodiment of the present disclosure is directed to providing a kit for detecting functional metabolite biosynthetic strains comprising a composition for detecting functional metabolite biosynthetic strains.

Further, still another embodiment of the present disclosure is directed to providing a method for detecting functional metabolite biosynthetic strains under isothermal one-pot reaction conditions.

Terms used herein are solely used for illustration purposes, and should not be construed as limiting the present disclosure. Singular expressions shall include plural expressions unless the context clearly indicates otherwise. In the present specification, terms such as “comprise” or “have” are intended to designate the presence of features, numbers, steps, operations, components, parts, or combinations thereof as described in the specification without precluding the possibility of the presence or addition of one or more other features, numbers, steps, operations, components, parts, or combinations thereof.

Further, unless defined otherwise, all terms used herein, including technical or scientific terms, have the same meaning as commonly understood by one of ordinary skill in the art to which the embodiments belong. Terms such as those defined in commonly used dictionaries should be interpreted as having a meaning consistent with the meaning in the context of the related art, and unless explicitly defined in the present application, it should not to be construed in an idealized or overly formal sense.

Hereinafter, the present disclosure will be described in more detail.

The principle of the isothermal one-pot reaction probe set according to the present disclosure may be confirmed by the previously developed RNA detection technology [Sensitive splint-based one-pot isothermal RNA detection (SENSR); hereinafter SENSR technology; Nat Biomed Eng 4, 1168-1179 (2020)], which is described in detail as follows.

The present disclosure has an isothermal one-pot reaction probe set for detecting a target nucleic acid sequence including a first probe and a second probe, which is used for the detection of a functional metabolite biosynthetic strain.

The first probe is a promoter probe (PP) having a structure of the following general formula (I);

3′-X-Y-5′ (I)
in the general formula (I) above,
X is an upstream hybridization sequence (UHS) portion having a hybridization sequence complementary to the target nucleic acid sequence; the target nucleic acid sequence is DNA or RNA; Y is a stem-loop structure portion containing a promoter sequence recognizable by an RNA polymerase; and X and Y are deoxyribonucleotides; and the second probe is a reporter probe (RP) having a structure of the following general formula II;

3′-Z-X′-5′ (II)
in the general formula (II) above,
Z is an aptamer sequence portion with an interactive labeling system comprising one label or a plurality of labels generating a detectable signal; the target nucleic acid sequence is DNA or RNA; X′ is a downstream hybridization sequence (DHS) portion having a hybridization sequence complementary to the target nucleic acid sequence; and Z and X′ are deoxyribonucleotides.

The first probe and the second probe are hybridized to the target nucleic acid sequence and ligated; and the thus-obtained ligation product is transcribed by an RNA polymerase to generate a signal.

More specifically, each component is described as follows.

The first probe of the probe set of the present disclosure has a structure comprising 25 unique different portions, each within one oligonucleotide molecule:

X which is an upstream hybridization sequence (UHS) portion having a hybridization sequence complementary to the target nucleic acid sequence; and Y which is a stem-loop structure portion containing a promoter sequence recognizable by an RNA polymerase.

Further, the second probe of the probe set of the present disclosure has a structure comprising unique different portions, each within one oligonucleotide molecule:

Z which is an aptamer sequence portion with an interactive labeling system comprising one label or a plurality of labels generating a detectable signal; X′ is a downstream hybridization sequence (DHS) portion having a hybridization sequence complementary to the target nucleic acid sequence.

This structure enables the probe set of the present disclosure to become a probe that exhibits a high detection effect in a short time under isothermal one-pot reaction conditions as a unified step.

More specifically, the probe design of the present disclosure allows two single-stranded DNA probes to hybridize the probe set with the target RNA/DNA. The hybridization sequences are designed to maximize hybridization to the target RNA/DNA while minimizing the formation of any other structures. An efficient hybridization process between the probe and target RNA/DNA allows for high sensitivity during isothermal reactions.

The promoter probe which is the first probe is designed to form a stem-loop structure, and the stem portion forms a double-stranded RNA polymerase promoter sequence to initiate a transcription process using RNA polymerase. Since the double-stranded RNA polymerase promoter portion is physically linked by the loop sequence, the probability that the double-stranded promoter is formed into a functional form is higher than when the double-stranded promoter is not linked by the loop sequence. Therefore, the self-assembled promoter sequence in which the hairpin structure is formed in the promoter probe may effectively promote the hybridization process and the subsequent transcription process.

The reporter probe which is the second probe is designed to include an aptamer sequence as a reporter, and the final product may be identified by the aptamer that generates a signal in response to a specific substance.

In the present disclosure, “aptamer” used as a reporter is a single-stranded nucleic acid (DNA, RNA or modified nucleic acids) that has a stable tertiary structure and is capable of binding to a target molecule with high affinity and specificity.

As long as the aptamer of the present disclosure and the reaction material interacting with the aptamer generate a detectable signal as a target effect, any kind of aptamer and reaction material may be used.

After hybridization, the first probe and the second probe hybridized to the target nucleic acid sequence are ligated. In other words, after the first probe and the second probe of the present disclosure are hybridized with the target nucleic acid sequence, a nick formed between the two probes is ligated using a ligation agent.

According to a preferred embodiment of the present disclosure, the first probe and the second probe are positioned at immediately adjacent locations to each other when hybridized with the target nucleic acid sequence, respectively. The adjacent positioning is necessary for ligation reactions between the two probes. The term used herein “adjacent” in conjunction with hybridization positions of the first probe and the second probe means that the 3′-end of one probe and the 5′-end of the other probe are sufficiently close to each other to allow connection of the ends of both probes to one another.

Since enzymatic ligation is the preferred method of linking the first probe and the second probe by a covalent bond, the term “ligation” is used throughout this specification. The term “ligation” is a general term and is to be understood to include any method of linking two probes by a covalent bond.

The ligation reaction of the present disclosure may be performed using a wide variety of ligation agents, including enzymatic ligation agents and non-enzymatic ligation agents (e.g., chemical agents and photoligation agents).

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light.

Autoligation, i.e., spontaneous ligation in the absence of a ligating agent, is also within the scope of the present disclosure.

Photoligation using light of an appropriate wavelength as the ligation agent is also within the scope of the present disclosure. According to certain embodiments, the photoligation comprises probes comprising nucleotide analogs, including but not limited to, 4-thiothymidine (s4T), 5-vinyluracil and its derivatives, or combinations thereof.

According to a preferred embodiment of the present disclosure, the ligation reaction is performed by an enzymatic ligation agent, wherein the ligation agent includes one selected from the group consisting of SplintR ligase, bacteriophage T4 ligase, E. coli ligase, Afu ligase, Taq ligase, Tfl ligase, Mth ligase, Tth ligase, Tth HB8 ligase, Thermus species AK16D ligase, Ape ligase, LigTk ligase, Aae ligase, Rm ligase, Pfu ligase, ribozyme and variants thereof.

The internucleotide linkage generated by the ligation includes phosphodiester bond and other linkages. For instance, the ligation using ligases generally produces phosphodiester bonds.

Non-enzymatic methods for ligation may form other internucleotide linkages. Other internucleotide linkages include, without limitation, covalent bond formation between appropriate reactive groups such as between an x-haloacyl group and a phosphothioate group to form a thiophosphorylacetylamino group, a phosphorothioate and tosylate or iodide group to form a 5′-phosphorothioester, and pyrophosphate linkages.

After the ligation reaction, the resulting ligation product includes an aptamer sequence and becomes single-stranded DNA as a template for amplifying the target RNA/DNA.

Subsequently, when the transcriptional process is initiated by RNA polymerase, the target RNA/DNA is elongated from the single-stranded DNA, which is a template for amplifying the target RNA/DNA containing the aptamer sequence such that signals are generated quickly and simply by the aptamer introduced to fluoresce by binding to specific chemical molecules.

Further, when the RNA aptamer is used as a reporter, the time it takes to observe the generated signal is shortened, compared to a conventional fluorescent signal using a fluorescent protein.

If the first probe and the second probe are not performed as described above, a signal emitted from the label on the transcriptional product of the first probe and the second probe is not finally generated, and thus the target nucleic acid sequence is not detected.

The RNA polymerase of the present disclosure may include any kind of RNA polymerase as long as it can recognize the promoter portion so as to initiate transcription as the desired effect.

Preferably, the RNA polymerase may be selected from the group consisting of bacteriophage T7 RNA polymerase, bacteriophage T3 polymerase, bacteriophage RNA polymerase, bacteriophage @II polymerase, Salmonella bacteriophage sp6 polymerase, Pseudomonas bacteriophage gh-1 polymerase, E. coli RNA polymerase holoenzyme, E. coli RNA polymerase core enzyme, human RNA polymerase I, human RNA polymerase II, human RNA polymerase III, human mitochondrial RNA polymerase and variants thereof, but is not limited thereto.

Likewise, as long as transcription may be initiated by RNA polymerase, any promoter sequence known in the art may be employed for the promoter region in the first probe of the present disclosure, which is recognized by RNA polymerase.

The ligation agent and the polymerase may be appropriately adjusted to optimize the isothermal one-pot reaction of the present disclosure. They are included preferably 1:1 to 1:5 units, more preferably 1:4 units, most preferably 1:2 units in a one-pot reaction buffer.

In the present disclosure, the label may be any one selected from the group consisting of a chemical label, an enzyme label, a radioactive label, a fluorescent label, a luminescent label, a chemiluminescent label, and a metal label.

The isothermal one-pot reaction of the present disclosure is performed simultaneously, without a separate amplification reaction, unified by any one specified temperature in the range of 15° C. to 50° C. The temperature in the range of 15° C. to 50° C. is a temperature known in the art at which enzymes act, and is not limited thereto, as long as the desired effect of the present disclosure may be obtained.

In an embodiment of the present disclosure, the specified temperature is preferably 37° C.

The isothermal one-pot reaction is performed simultaneously, unified with a one-pot reaction buffer containing Tris-HCl, MgCl₂, NTPs, and extreme thermostable single-stranded DNA binding protein (ET-SSB).

In the present t disclosure, the one-pot reaction buffer preferably includes 1 to 500 mM Tris-HCl; 1 to 200 mM MgCl₂; 0.1 to 50 mM NTPs; and 1 to 800 ng ET-SSB, more preferably 100 to 400 mM Tris-HCl; 50 to 150 mM MgCl₂; 10 to 40 mM NTPs; and 100 to 600 ng ET-SSB, and the most preferably, 350 mM Tris-HCl; 100 mM MgCl₂; 25 mM NTPs; and 500 ng ET-SSB.

In the present disclosure, the functional metabolite may preferably be at least any one selected from the group consisting of carotenoid, 1-deoxynojirimycin (DNJ), violacein, staurosporine, and rebeccamycin.

In the present disclosure, the target nucleic acid sequence may preferably be DNA or RNA sequence of at least any one gene selected from the group consisting of gabT1, yktc1, gutB1, vioA, vioB, vioE, vioD, vioC, staP, staG, staN, staMA, staMB, rebH, rebo, rebD, rebC, rebP, rebG, rebM, crtB, crtI, lyeJ, cruF, crtEb, cruY, crtM, crtN, crtP, crtQ, and crtO.

In the present disclosure, the probe set may preferably be a probe set for detecting any one strain selected from the group consisting of the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus, and Lentzea aerocolonigenes.

There are many different types of carotenoids produced by strains, which may be categorized by the length of carbon backbone thereof. More specifically, 4,4′-diaponeurosporene, known as a C30 carotenoid produced by lactic acid bacteria, is biosynthesized from FPP by dehydrosqualene desaturase (CrtN) and dehydrosqualene synthase (CrtM), but since these biosynthetic genes are strain-specific and localized to a limited number of strains, it is preferred in the probe set of the present disclosure that the target nucleic acid sequence is DNA or RNA sequence of the crtM gene or the crtN gene. Here, 4,4′-diaponeurosporene may preferably be biosynthesized from lactic acid bacteria of the genus Lactiplantibacillus, the genus Enterococcus and the genus Leuconostoc.

Further, staphyloxanthin, known as a C30 carotenoid, is biosynthesized by crtM, crtN, crtP, crtQ, and crtO, but since these biosynthetic genes are strain-specific and localized to a limited number of strains, it is preferred in the probe set of the present disclosure that the target nucleic acid sequence is DNA or RNA sequence of any one gene selected from the group consisting of crtM, crtN, crtP, crtQ, and crtO. In this case, the staphyloxanthin may preferably be biosynthesized from Staphylococcus aureus.

In other words, the probe set according to the present disclosure may be constructed on the basis of DNA or RNA sequences of crtM, crtN, crtP, crtQ and crtO of strains known to produce C30 carotenoids, and thus the strains capable of being detected may be, without limitation, strains known to produce C30 carotenoids. Preferably, the probe set may be used to detect any one strain selected from the group consisting of the genus Lactiplantibacillus, the genus Enterococcus, the genus Leuconostoc, and Staphylococcus aureus.

Here, the genus Lactiplantibacillus may comprise Lactiplantibacillus plantarum, and the genus Enterococcus may comprise at least any one selected from the group consisting of Enterococcus casseliflavus, Enterococcus faecalis, Enterococcus faecium, Enterococcus gallinarum, Enterococcus mundtii, and Enterococcus saccharolyticus. Further, the genus Leuconostoc may comprise, but is not limited to, Leuconostoc citreum.

Here, in the genus Enterococcus, the nucleotide sequence of the C30 carotenoid biosynthetic cluster is significantly different depending on the species. Therefore, it is more preferable that the probe set for the detection of Enterococcus gilvus (E. gilvus) among strains belonging to the genus Enterococcus is generally designed to be different from the probe set for species belonging to the genus Enterococcus.

In this case, in the genus Leuconostoc, the nucleotide sequence of the C30 carotenoid biosynthetic cluster is significantly different depending on the species. Therefore, it is more preferable that the probe set for the detection of Leuconostoc mesenteroides (L. mesenteroides) among strains belonging to the genus Leuconostoc is generally designed to be different from the probe set for species belonging to the genus Leuconostoc.

More specifically, a composition for detecting C30 carotenoid biosynthetic strains comprising: an isothermal one-pot reaction probe set for detecting the genus Lactiplantibacillus including a first probe and a second probe is designed to have the first probe of SEQ ID NO: 1 or 3 and the second probe of SEQ ID NO: 2 or 4.

More specifically, a composition for detecting C30 carotenoid biosynthetic strains comprising: a probe set for detecting the genus Enterococcus including a first probe and a second probe may have the first probe of SEQ ID NO: 5 and the second probe of SEQ ID NO: 6.

More specifically, a composition for detecting C30 carotenoid biosynthetic strains comprising: a probe set for detecting Enterococcus gilvus including a first probe and a second probe may have the first probe of SEQ ID NO: 7 and the second probe of SEQ ID NO: 8.

More specifically, a composition for detecting C30 carotenoid biosynthetic strains comprising: a probe set for detecting the genus Leuconostoc including a first probe and a second probe may have the first probe of SEQ ID NO: 9 and the second probe of SEQ ID NO: 10.

More specifically, a composition for detecting C30 carotenoid biosynthetic strains comprising: a probe set for detecting Leuconostoc mesenteroides including a first probe and a second probe may have the first probe of SEQ ID NO: 11 and the second probe of SEQ ID NO: 12.

More specifically, the SEQ ID NOS: 1, 3, 5, 7, 9, and 11 comprise a hybridization sequence complementary to the target nucleic acid sequence and a T7 promoter complementary sequence+a loop sequence+a T7 promoter sequence, and the SEQ ID NOs: 2, 4, 6, 8, 10 and 12 comprise an aptamer sequence and a hybridization sequence complementary to the target nucleic acid sequence.

The probe set according to the present disclosure may be constructed on the basis of DNA or RNA sequences of crtB or crtI of strains known to produce C40 carotenoids, and thus the strains capable of being detected may be, without limitation, strains known to produce C40 carotenoids. Preferably, the probe set may be used to detect strains of the genus Rhodococcus or the genus Gordonia.

Further, bacterioruberin, known as a C50 carotenoid, is biosynthesized by CrtB, CrtI, LyeJ, and CruF, but since these biosynthetic genes are strain-specific and localized to a limited number of archaea, it is preferred in the probe set of the present disclosure that the target nucleic acid sequence is DNA or RNA sequence of any one gene selected from the group consisting of crtB, crtI, lyeJ, and cruF.

The probe set according to the present disclosure may be constructed on the basis of DNA or RNA sequences of any one gene selected from the group consisting of crtB, crtI, lyeJ, and cruF of strains known to produce C50 carotenoids, and thus the strains capable of being detected may be, without limitation, strains known to produce C50 carotenoids. Preferably, the probe set may be used to detect strains of the genus Haloferax.

Further, decaprenoxanthin, known as a C50 carotenoid, is biosynthesized by CrtB, CrtI, CrtEb, and CruY, but since these biosynthetic genes are strain-specific and localized to a limited number of archaea, it is preferred in the probe set of the present disclosure that the target nucleic acid sequence is DNA or RNA sequence of any one gene selected from the group consisting of crtB, crtI, crtEb, and cruY.

The probe set according to the present disclosure may be constructed on the basis of DNA or RNA sequences of any one gene selected from the group consisting of crtB, crtI, crtEb, and cruY of strains known to produce C50 carotenoids, and thus the strains capable of being detected may be, without limitation, strains known to produce C50 carotenoids. Preferably, the probe set may be used to detect strains of the genus Arthrobacter.

Meanwhile, 1-deoxynojirimycin, DNJ, one of the functional metabolites known to have antidiabetic activity, is biosynthesized by GabT1, Yktc1, and GutB1, but since these biosynthetic genes are strain-specific and localized to a limited number of strains, it is preferred in the probe set of the present disclosure that the target nucleic acid sequence is DNA or RNA sequence of at least any one gene selected from the group consisting of gabT1, yktc1, and gutB1.

The probe set according to the present disclosure may be constructed on the basis of DNA or RNA sequences of at least any one gene selected from the group consisting of gabT1, yktc1, and gutB1 of strains known to produce 1-deoxynojirimycin, and thus the strains capable of being detected may be, without limitation, strains known to produce 1-deoxynojirimycin. Preferably, the probe set may be used to detect strains of the genus Bacillus.

In this case, the strain of the genus Bacillus may be any one selected from the group consisting of, for example, Bacillus subtillis, Bacillus atrophaeus, Bacillus velezensis, and Bacillus amyloliquefaciens.

Meanwhile, violacein, one of the functional metabolites known to have antibacterial, antiparasitic, and anticancer activity, is biosynthesized by VioA, VioB, VioE, VioD, and VioC, but since these biosynthetic genes are strain-specific and localized to a limited number of strains, it is preferred in the probe set of the present disclosure that the target nucleic acid sequence is DNA or RNA sequence of at least any one gene selected from the group consisting of vioA, vioB, vioE, vioD, and vioC.

The probe set according to the present disclosure may be constructed on the basis of DNA or RNA sequences of at least any one gene selected from the group consisting of vioA, vioB, vioE, vioD, and vioC of strains known to produce violacein, and thus the strains capable of being detected may be, without limitation, strains known to produce violacein. Preferably, the probe set may be used to detect Chromobacterium violaceum.

Meanwhile, staurosporine, one of the functional metabolites known to have anticancer activity, is biosynthesized by StaP, StaG, StaN, StaMA, and StaMB, but since these biosynthetic genes are strain-specific and localized to a limited number of strains, it is preferred in the probe set of the present disclosure that the target nucleic acid sequence is DNA or RNA sequence of at least any one gene selected from the group consisting of staP, staG, staN, staMA, and staMB.

The probe set according to the present disclosure may be constructed on the basis of DNA or RNA sequences of at least any one gene selected from the group consisting of staP, staG, staN, staMA, and staMB of strains known to produce staurosporine, and thus the strains capable of being detected may be, without limitation, strains known to produce staurosporine. Preferably, the probe set may be used to detect Streptomyces staurosporeus.

Meanwhile, rebeccamycin, one of the functional metabolites known to have antitumor activity, is biosynthesized by RebH, RebO, RebD, RebC, RebP, RebG, and RebM, but since these biosynthetic genes are strain-specific and localized to a limited number of strains, it is preferred in the probe set of the present disclosure that the target nucleic acid sequence is DNA or RNA sequence of at least any one gene selected from the group consisting of rebH, rebo, rebD, rebC, rebP, rebG, and rebM.

The probe set according to the present disclosure may be constructed on the basis of DNA or RNA sequences of at least any one gene selected from the group consisting of rebH, rebo, rebD, rebC, rebP, rebG, and rebM of strains known to produce rebeccamycin, and thus the strains capable of being detected may be, without limitation, strains known to produce rebeccamycin. Preferably, the probe set may be used to detect Lentzea aerocolonigenes.

In particular, compositions for detecting the above-described functional metabolite biosynthetic strains according to the present disclosure (Specifically, composition for detecting the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus and Lentzea aerocolonigenes) may be used by mixing two or more compositions. By using this, it is possible to perform rapid and accurate detection on the same functional metabolite biosynthetic strains or different functional metabolite biosynthetic strains in different genera.

The detection of these functional metabolite biosynthetic strains may also be applied in a multiplexed manner. As suitable fluorescent probes, aptamers may be used in combination with compositions for detection of two or more functional metabolite biosynthetic strains, for example, by combining an aptamer specific for broccoli with an aptamer specific for malachite green.

Specifically, the spectral range of malachite green is emission: 616 nm, excitation: 665 nm, and the spectral range of broccoli aptamer is emission: 460 nm, excitation: 520 nm, and thus compositions for detecting the same functional metabolite biosynthetic strains or different functional metabolite biosynthetic strains in two or more different genera may also be used together to have the advantage of detecting functional metabolite biosynthetic strains of various genera or species at once.

The probe set of the present disclosure may serve as a powerful diagnostic platform for detecting RNA of the target to not only provide a short diagnosis time, high sensitivity and specificity and a simple analysis procedure, but also does not require expensive equipment and diagnostic experts, thereby making it possible to be used as a suitable method for rapid detection of functional metabolite biosynthetic strains.

According to still another aspect of the present disclosure, the present disclosure provides a method for detecting functional metabolite biosynthetic strains under isothermal one-pot reaction conditions, comprising the following steps.

Matters described in the composition are not set forth herein to avoid redundancy, but are equally applicable to matters related to the methods described herein.

The functional metabolite may preferably be at least any one selected from the group consisting of carotenoid, 1-deoxynojirimycin (DNJ), violacein, staurosporine, and rebeccamycin.

Matters as to the compositions for detecting functional metabolite biosynthetic strains (Specifically, composition for detecting the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus and Lentzea aerocolonigenes) are also applied to the method below, and detailed description thereof is omitted.

The method for detecting functional metabolite biosynthetic strains as described above, specifically, comprises:
(a) treating a sample with an isothermal one-pot reaction probe set for detecting a functional metabolite biosynthetic strain including a first probe and a second probe; and hybridizing the probe set to a target nucleic acid (preferably RNA) sequence;
(b) treating the hybridization product of step (a) above with a ligation agent to ligate the first and second probes of the probe set, and treating the thus-obtained ligation product with a polymerase to initiate transcription; and
(c) treating the transcription product of step (b) with an aptamer-reaction material to detect signal generation of an aptamer in the transcription product.

Matters as to the functional metabolite biosynthetic strains may be applied, respectively, to the detection of strains of the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus, and Lentzea aerocolonigenes or to strains known to be functional metabolite biosynthetic strains.

The target sequence may be DNA or RNA sequence of at least any one gene selected from the group consisting of gabT1, yktc1, gutB1, vioA, vioB, vioE, vioD, vioC, staP, staG, staN, staMA, staMB, rebH, rebo, rebD, rebC, rebP, rebG, rebM, crtB, crtI, lyeJ, cruF, crtEb, cruY, crtM, crtN, crtP, crtQ, and crtO.

Meanwhile, when the same functional metabolite biosynthetic strains or different functional metabolite biosynthetic strains in different genera described above are attempted to be detected in a single reaction, it is possible to perform detection in one pot by varying the fluorescence capable of detecting the signal generation of the aptamer.

The isothermal one-pot reaction of the present disclosure is performed simultaneously, without a separate amplification reaction, unified by any one of the specified temperatures in the range of 15° C. to 50° C., and preferably, the specified temperature is 37° C.

The isothermal one-pot reaction is performed simultaneously, unified with a one-pot reaction buffer containing Tris-HCl, MgCl₂, NTPs, and extreme thermostable single-stranded DNA binding protein (ET-SSB).

In addition, the present disclosure is characterized in that an additional separate amplification process (e.g., PCR) is not required, and the amplification process is performed automatically during the isothermal one-pot reaction.

The feasibility of the isothermal one-pot reaction including an amplification process is achieved because of (i) the design of the second probe to form an aptamer downstream due to a ligation reaction occurring with the first probe including the double-stranded promoter sequence having a hairpin structure; and (ii) the amplification process that occurs naturally in the reaction without a separate amplification process, by using what may be used as a target sequence (i.e., splint) when the ligated product is transcribed by transcription initiation.

That is, when the ligation product of the ligation reaction between the first probe and the second probe is subjected to transcription initiation from the RNA polymerase promoter sequence of the first probe that forms a hairpin structure and a transcript is then produced, the transcript contains the same sequence as the target RNA sequence to thereby be usable as the target RNA. In addition, since the transcribed ligation product includes the same sequence as the target nucleic acid sequence, it may act as a target RNA among target nucleic acids.

Therefore, the platform using the probe set of the disclosure is designed to allow ligation, present transcription reaction, and fluorescence reaction to occur simultaneously, which is an isothermal one-pot reaction in which the transcript formed by the transcription reaction from the ligated product is used as a splint RNA, thereby enabling the amplification process to be included.

Accordingly, steps (a) to (c) of the present disclosure may be performed simultaneously in one vessel, such as a tube.

In the present disclosure, the sequences of the DNA probe are not limited to the sequences described in the Examples of the present disclosure as long as the desired effect is achieved, but are applicable to all target gene sequences. In addition, the aptamer used for final signal confirmation is not limited to the malachite green aptamer, and may be any type of RNA-based fluorescent aptamer.

According to an embodiment of the present disclosure, the method according to the present disclosure has the advantage that the design of the probe set specific to the functional metabolite biosynthetic strain as described above enables the isolation of the functional metabolite biosynthetic strain as well as the identification of the strain.

Furthermore, by utilizing the probe set as described above, the method according to the present disclosure is able to also detect RNA of essential biosynthetic gene clusters for the detection of rare strains producing functional metabolite.

In particular, the isothermal one-pot reaction according to the present disclosure has the advantage of enabling the isolation of functional microorganisms capable of producing useful active substances through the isothermal one-pot reaction in a strain lysate, without the need to purify the nucleic acids of the strain.

Since the method of the present disclosure encompasses the probe set of the present disclosure described above, the overlapping contents are excluded in order to avoid the complexity of the present specification.

In another embodiment of the present disclosure, the present disclosure provides a kit for detecting functional metabolite biosynthetic strains, comprising: an isothermal one-pot reaction probe set for detecting a functional metabolite biosynthetic strain including the first and second probes described above; a ligation agent; a polymerase; and an isothermal one-pot reaction buffer.

According to another aspect of the present disclosure, the present disclosure provides a kit for detecting functional metabolite biosynthetic strains comprising a composition for detecting a functional metabolite biosynthetic strain as described above; a ligation agent; a polymerase; and an isothermal one-pot reaction buffer.

Here, the functional metabolite may preferably be at least any one selected from the group consisting of carotenoid, 1-deoxynojirimycin (DNJ), violacein, staurosporine, and rebeccamycin.

Here, the functional metabolite biosynthetic strain may be the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus, and Lentzea aerocolonigenes, but is not necessarily limited thereto.

The kit may comprise two or more targeted functional metabolite biosynthetic strain probe sets. In this case, the probe sets each comprise different interactive labeling systems; and each of the probe sets binds to a different target nucleic acid sequence; enabling multiple detection of different target nucleic acid sequences. In other words, it is possible to simultaneously detect the same functional metabolite biosynthetic strains or different functional metabolite biosynthetic strains in different genera.

Further, the two or more types of targets may be different target portions present in functional metabolite biosynthetic strains of different genera or species, and in this case, different portions of the target are effectively detected to enable a more accurate and precise diagnosis.

The kit of the present disclosure is manufactured to perform the detection of a target nucleic acid sequence by the probe set of the present disclosure as described above. Thus, the overlapping contents are excluded in order to avoid the complexity of the present specification.

The kit of the present disclosure as described above may additionally include various polynucleotide molecules, enzymes, various buffers and reagents. Further, the kit of the present disclosure may include reagents essential for performing positive control and negative control reactions. The optimal amount of reagent to be used in any one particular reaction may be easily determined by those skilled in the art having learned the disclosure herein.

Typically, the kit of the present disclosure is manufactured in a separate package or compartment including the above described components.

### BRIEF DESCRIPTION OF THE DRAWINGS

FIG. 1 is a schematic view illustrating structures of DNA probes according to the present disclosure.

FIG. 2 shows screening results based on the phenotype of strains upon isolation.

FIG. 3 shows the results confirming whether the strains isolated through primary screening are lactic acid bacteria.

FIG. 4 shows the experimental results in which four randomly selected strains are subjected to an isothermal one-pot reaction, using a set of single-stranded, isothermal one-pot reaction probes mixed in strain lysates.

FIG. 5 shows the confirmation of the C30 carotenoid biosynthetic gene cluster in four strains after the isothermal one-pot reaction.

FIG. 6 is a phylogenetic tree representing the results of the identification of four strains after the isothermal one-pot reaction.

### DETAILED DESCRIPTION OF EMBODIMENTS

Hereinafter, the present disclosure will be described in more detail through Examples. These Examples are provided solely for the purpose of illustrating the present disclosure, and it will be apparent to those skilled in the art that the scope of the present disclosure should not be construed as limited by these Examples.

### Experimental Materials

All kimchi used in the present experiment was provided from home. MRS (De Man, Rogosa, and Sharpe) medium and PCA medium supplemented with BCP (Bromocresol Purple) used for isolation of lactic acid bacteria were purchased from MBcell (Seoul, Republic of Korea).

All single-stranded DNA used as probes were custom-made by Bioneer, and the reaction buffer for the isothermal single reaction, Tris-HCl (pH 7.4) and 1 M MgCl₂, were purchased from Bioneer, Inc (Daejeon, Republic of Korea). In addition, SplintR ligase, T7 RNA polymerase, and extreme thermostable single-stranded DNA binding protein (ET-SSB) were purchased from New England Biolabs (NEB, Ipswich, MA, USA). Further, s purchased from Thermo Fisher Scientific (Waltham, MA, USA), and Recombinant RNase Inhibitor (RRI) was purchased from Takara (Kyoto, Japan). Malachite green oxalate, a fluorescent dye, was purchased from Sigma-Aldrich (St. Louis, MO, USA).

Q5 polymerase used in the PCR was purchased from NEB (Ipswich, MA, USA), dNTPs were purchased from Takara (Kyoto, Japan), and primers were synthesized by Cosmogenetech, Inc. (Seoul, Republic of Korea). The kits used for DNA extraction and purification of PCR products were purchased from GeneAll Biotechonology (Seoul, Republic of Korea).

### Example 1. Design and Construction of C30 Carotenoid-Specific DNA Probe

### A. Design of C30 Carotenoid-Specific DNA Probe

The essential biosynthetic genes for C30 carotenoid production in lactic acid bacteria are reported to be crtN and crtM genes, with the enzyme encoded by the crtN gene being dehydrosqualene desaturase and the enzyme encoded by the crtM gene being dehydrosqualene synthase. However, in some cases, phytoene desaturase and phytoene synthase, which are C40 carotenoid biosynthesis genes, are used interchangeably as crtN and crtM, respectively. Therefore, the crtN and crtM genes were searched in the NCBI database for obtaining genetic sequence information, to confirm information regarding lactic acid bacteria in which each gene was reported.

Strains containing the C30 carotenoid biosynthetic cluster reported in the NCBI were confirmed as Leuconostoc Lactiplantibacillus plantarum, citreum, Leuconostoc mesenteroides, Enterococcus casseliflavus, Enterococcus faecalis, Enterococcus faecium, Enterococcus gilvus, Enterococcus gallinarum, Enterococcus mundtii, and Enterococcus saccharolyticus. Of these, Lactiplantibacillus plantarum was the most commonly reported, with 44 cases.

First, DNA probes were designed to detect the C30 carotenoid biosynthetic cluster for each genus. For the design, NCBI's primer-BLAST was used with the following conditions in which the PCR product size was set to 40-60 nt and the primer size was set to 15-25 nt. The reverse primer of the retrieved primer was then designed as the UHS (upstream hybridization sequence; 3′-5′) of the promoter probe, and the DHS (downstream hybridization sequence; 3′-5′) was designed by selecting the sequence with the most similar Tm value to the UHS in the 5′ direction of the UHS. Here, the selection of the DNA probe was based on the formation of aptamers capable of expressing fluorescence in the reporter probe. The aptamer was a malachite green aptamer with an optimal wavelength band.

### B. Construction of C30 Carotenoid-Specific DNA Probe

The formation of aptamers was confirmed with the NUPACK program for the construction of the previously selected DNA probe.

Among 38 candidate DNA probes for the detection of the crtM gene in the genus Lactiplantibacillus, the aptamer formation was confirmed in 7 DNA probes, and among 29 candidate DNA probes for the detection of the crtN gene, the aptamer formation was confirmed in 4 DNA probes.

In the genus Enterococcus, the nucleotide sequences of the C30 carotenoid biosynthetic cluster are significantly different depending on the species. Therefore, crtM gene-targeted DNA probes for detection of Enterococcus casseliflavus (E. casseliflavus), Enterococcus faecalis (E. faecalis), and Enterococcus faecium (E. faecium) and crtN gene-targeted DNA probe for detection of Enterococcus gilvus (E. gilvus) were designed, respectively. As a result, the aptamer formation was confirmed in 7 out of 22 candidate DNA probes for detection of the crtM gene, and in 9 out of 20 candidate DNA probes for detection of the crtN gene.

As to the genus Leuconostoc, for the same reasons as to the genus Enterococcus, crtM gene-targeting DNA probe for detection of Leuconostoc. citreum and crtN gene-targeting DNA probe for detection of Leuconostoc. mesenteroides, were designed, respectively. As a result, the aptamer formation was confirmed in 12 out of 40 candidate DNA probes for detection of the crtM gene, and in 6 out of 29 candidate DNA probes for detection of the crtN gene.

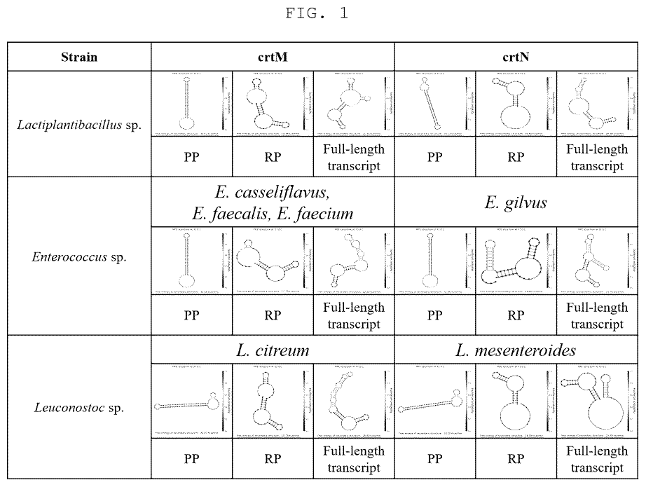

From each DNA probe set in which aptamer formation was confirmed as above, the DNA probe set for which the most stable aptamer formation was confirmed was selected, and finally, six DNA probe sets specific for each strain were constructed as shown in FIG. 1.

To implement this concept, previously developed RNA detection technology [Sensitive splint-based one-pot isothermal RNA detection (SENSR); hereinafter, referred to as SENSR technology; Nat Biomed Eng 4, 1168-1179 (2020)] was used to design the corresponding probe set, and the information is shown in Table 1 below.

TABLE 1

| Types of | | | |
|---|---|---|---|
| lactic | | | |
| acid | Gene | | |
| bacteria | type | Type | Sequence information (5′-3′) |
| Lactiplanti- | crtM | PP | SEQ ID NO: 1 |
| bacillus | | | [phosphate]ACGATAAAGCAAAGTGGCGGccctatagt |
| sp. | | | gagtcgtattaatttcgcgacaacacgcgaaattaatacg |
| | | | actcactataggg |
| | | RP | SEQ ID NO: 2 |
| | | | ggatccattcgttacctggctctcgccagtcgggatccTT |
| | | | CACGTGCGATTAGCAATAATTC |
| | crtN | PP | SEQ ID NO: 3 |
| | | | [phosphate]AGTCACTGTGGATCGGATGAccctatagt |
| | | | gagtcgtattaatttcgcgacaacacgcgaaattaatacg |
| | | | actcactataggg |
| | | RP | SEQ ID NO: 4 |
| | | | ggatccattcgttacctggctctcgccagtcgggatccGG |
| | | | AAAATCAGCATTACATAGGACAT |
| Enterococcus | crtM | PP | SEQ ID NO: 5 |
| sp. | | | [phosphate]TGGCTTCGATCAACAATGGGTccctatag |
| | | | tgagtcgtattaatttcgcgacaacacgcgaaattaatac |
| | | | gactcactataggg |
| | | RP | SEQ ID NO: 6 |
| | | | ggatccattcgttacctggctctcgccagtcgggatccGC |
| | | | ATTTCTTCCAAGCGTTTTTTCA |
| E. | crtN | PP | SEQ ID NO: 7 |
| gilvus | | | [phosphate]AAAGCAGCTCGATCATGGGAAccctatag |
| | | | tgagtcgtattaatttcgcgacaacacgcgaaattaatac |
| | | | gactcactataggg |
| | | RP | SEQ ID NO: 8 |
| | | | ggatccattcgttacctggctctcgccagtcgggatccTC |
| | | | CACCTTTCATAAAATGAATGCCAT |
| L. | crtM | PP | SEQ ID NO: 9 |
| citreum | | | [phosphate]GCCAACTATTGACCAACGCATccctatag |
| | | | tgagtcgtattaatttcgcgacaacacgcgaaattaatac |
| | | | gactcactataggg |
| | | RP | SEQ ID NO: 10 |
| | | | ggatccattcgttacctggctctcgccagtcgggatccTC |
| | | | GTGAGCAATCATTGCAGTCT |
| L. | crtN | PP | SEQ ID NO: 11 |
| mesenteroides | | | [phosphate]TTCGCAACTACGGCATCACAAccctatag |
| | | | tgagtcgtattaatttcgcgacaacacgcgaaattaatac |
| | | | gactcactataggg |
| | | RP | SEQ ID NO: 12 |
| | | | ggatccattcgttacctggctctcgccagtcgggatccTT |
| | | | GTCATTGTGTAAGGAAAATCAGCG |

In Table 1 above, capitalized nucleotides refer to the hybridization sequence complementary to the target nucleic acid sequence, lowercase nucleotides refer to the T7 promoter complementary sequence+loop sequence+T7 promoter sequence forming a stem-loop structure, and underlined nucleotides refer to the nucleotides forming the stem of the stem-loop structure. Nucleotides in lowercase italics are aptamer sequences. The [phosphate] indicates that it is phosphorylated at the 5′ end. In the probe set, PP/RP stands for promoter probe and reporter probe, respectively. The aptamer sequence is an aptamer that specifically binds to malachite green.

Meanwhile, the capitalized nucleotides above refer to the hybridization sequences complementary to the target nucleic acid sequence, specifically, the sequence numbers shown in Table 2 below.

TABLE 2

| Types of | | | |
|---|---|---|---|
| lactic acid | Gene | | |
| bacteria | type | Type | Sequence Information (5′→-3′) |
| Lactiplanti- | crtM | PP | SEQ ID NO: 13 |
| bacillus | | | ACGATAAAGCAAAGTGGCGG |
| sp. | | RP | SEQ ID NO: 14 |
| | | | TTCACGTGCGATTAGCAATAATTC |
| | crtN | PP | SEQ ID NO: 15 |
| | | | AGTCACTGTGGATCGGATGA |
| | | RP | SEQ ID NO: 16 |
| | | | GGAAAATCAGCATTACATAGGACAT |
| Enterococcus | crtM | PP | SEQ ID NO: 17 |
| sp. | | | TGGCTTCGATCAACAATGGGT |
| | | RP | SEQ ID NO: 18 |
| | | | GCATTTCTTCCAAGCGTTTTTTCA |
| E. gilvus | crtN | PE | SEQ ID NO: 19 |
| | | | AAAGCAGCTCGATCATGGGAA |
| | | RP | SEQ ID NO: 20 |
| | | | TCCACCTTTCATAAAATGAATGCCAT |
| L. citreum | crtM | PE | SEQ ID NO: 21 |
| | | | GCCAACTATTGACCAACGCAT |
| | | RP | SEQ ID NO: 22 |
| | | | TCGTGAGCAATCATTGCAGTCT |
| L. | crtN | PP | SEQ ID NO: 23 |
| mesenteroides | | | TTCGCAACTACGGCATCACAA |
| | | RP | SEQ ID NO: 24 |
| | | | TTGTCATTGTGTAAGGAAAATCAGCG |

### Example 2. Isolation of Lactic Acid Bacterial as Analysis Target

A. Isolation of Microorganisms from Fermented Food Samples

To isolate microorganisms, various types of kimchi were collected. A total of 27 types of kimchi were tested to isolate microorganisms, and even for the same kimchi, various microorganisms were isolated by differentiating the degree of fermentation, the region of manufacture, and the person who made the kimchi. Specifically, 17 types of napa cabbage kimchi, 3 types of radish kimchi, 2 types of young summer radish kimchi, 2 types of cucumber kimchi, 1 type of cabbage kimchi, 1 type of green onion kimchi, and 1 type of perilla leaf kimchi were tested for microbial isolation.

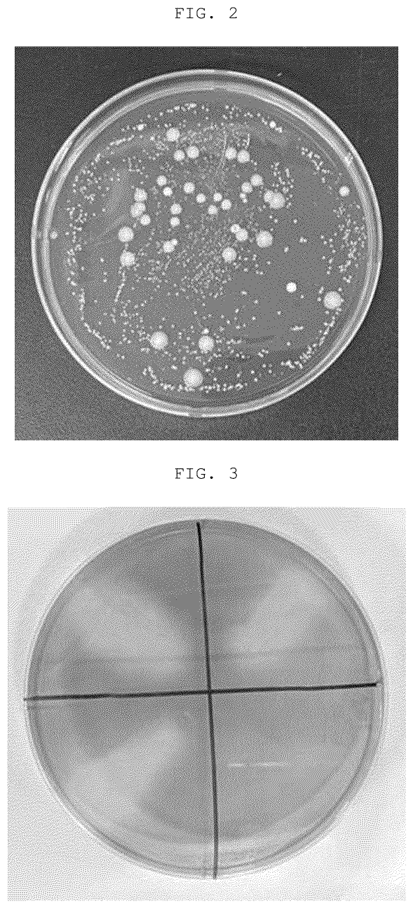

As shown in FIG. 2, MRS medium, a screening medium for lactic acid bacteria, was used as the isolation medium, and by varying the dilution factors of the medium, not only the dominant species but also the different species of microorganisms in the kimchi were attempted to be isolated. In other words, the strains were screened based on their phenotype (yellow colony formation).

### B. Isolation of Lactic Acid Bacteria as Analysis Target Among Isolated Microorganisms

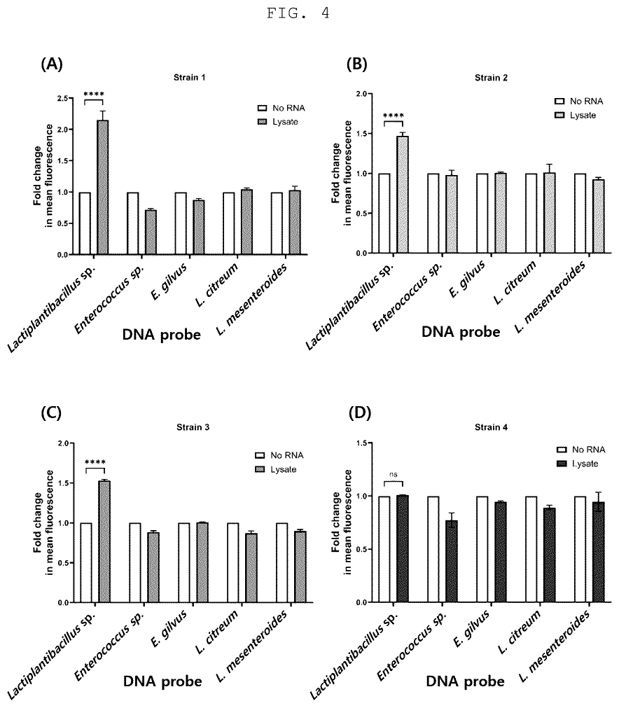

The microorganisms isolated above were first screened as general microorganisms and lactic acid bacteria based on the shape and size of the colonies and the presence or absence of EPS. The presumed lactic acid bacteria were then cultured on PCA medium supplemented with BCP, as shown in FIG. 3, and finally confirmed as lactic acid bacteria by the color change (yellow) of the medium. As a result, 645 lactic acid bacteria were isolated.

Most carotenoids are yellow or reddish in color, but most known carotenoids in lactic acid bacteria are yellow or beige in color, and thus a secondary screening was conducted based on the color phenotype (pigment) of the colonies exhibited by the strains. As a result, 411 lactic acid bacteria that exhibited yellow colonies were secured among 645 lactic acid bacteria.

### Example 3. Isothermal One-Pot Reaction with Probe Set for Detection of C30 Carotenoid-Biosynthetic Lactic Acid Bacteria

An isothermal one-pot reaction was performed to detect the C30 carotenoid biosynthetic gene on the lactic acid bacteria isolated in Example 2 above.

### A. Preparation of Lactic Acid Bacteria Lysate

The experiment was performed on four strains isolated from different kimchi samples. Each strain was dissolved in 1.5 ml of nuclease free water (NFW) and heated at 95° C. for 2 minutes to lysis, and then RNA was eluted.

### B. Confirmation of Detection of C30 Carotenoid Biosynthetic Lactic Acid Bacteria

Cell lysate, which was subjected to heat treatment only without any purification, was used as the target RNA for the isothermal one-pot reaction. Then, an isothermal one-pot reactant was prepared, which was reacted at 37° C. for 2 hours.

Table 3 below shows the reagent information of the one-pot reactant used in the experiments.

TABLE 3

| | | |
|---|---|---|
| Reagent | Concentration | Amount |

| | | | | |
|---|---|---|---|---|
| Promoter probe (PP) | 10 | mM | 1 | [symbol] |
| Reporter probe (RP) | 10 | mM | 2.2 | [symbol] |

| | | | |
|---|---|---|---|
| Cell lysate | | 3 | [symbol] |
| SENSR reaction buffer | 10X | 3 | [symbol] |
| NTPs | 25 mM each | 3 | [symbol] |

| | | | | |
|---|---|---|---|---|
| ATP | 1 | M | 0.3 | [symbol] |
| SplintR ligase | 25 | U/ [symbol] | 3 | [symbol] |
| T7 RNA polymerase | 50 | U/ [symbol] | 1.5 | [symbol] |
| Recombinant RNase | 40 | U/ [symbol] | 1 | [symbol] |

inhibitor

| | | | | |
|---|---|---|---|---|
| ET-SSB | 500 | ng/ [symbol] | 1 | [symbol] |
| Malachite green | 320 | mM | 1.5 | [symbol] |

| RNase-free water | up to 30 [symbol] | |
|---|---|---|
| Total | 30 | [symbol] |

The 10× SENSR reaction buffer from Table 3 above was composed of 350 mM Tris-HCl (pH 7.4) and 100 mM MgCl₂. Using the above one-pot reactants, isothermal one-pot reactions were performed, and then the presence or absence of the C30 carotenoid biosynthetic gene was confirmed by fluorescence measurements. Specifically, 20 μl of the reactant was dispensed into a 384-well clear flat-bottom black microplate, and the microplate was positioned in a microplate reader (Hidex Sense). Then, the fluorescence measurement was performed depending on the fluorescent dye to determine the presence or absence of fluorescence emission (ex: 616 nm/em: 665 nm for malachite green).

Isothermal one-pot reactions of C30 carotenoid-biosynthetic lactic acid bacteria were performed on the five pairs of probe sets designed in Example 1 above, and in the case of Lactiplantibacillus sp., the crtN DNA probe set was used (see Table 1). Each set of probes capable of detecting the C30 carotenoid cluster of each strain was reacted with the sample to confirm the presence of the corresponding sequence in the sample.

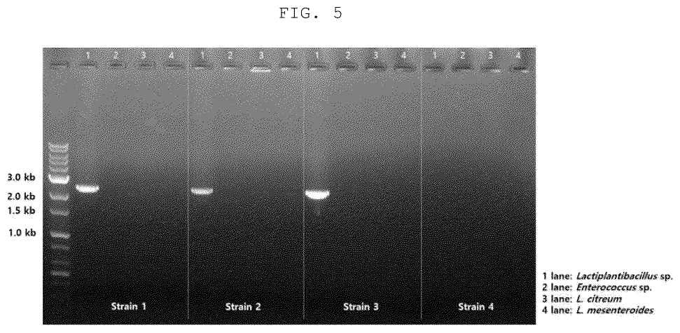

As can be seen in FIG. 4, in Strains 1, 2, and 3, the isothermal reaction performed with the probe set for detection of the C30 carotenoid biosynthetic essential gene of Lactiplantibacillus sp., showed an increase in fluorescence values. However, the isothermal reaction performed with the probe sets for the detection of the C30 carotenoid essential biosynthetic gene of Enterococcus sp., E. gilvus, Leuconostoc citreum, and Leuconostoc mesenteroides showed fluorescence values equal to or lower than the fluorescence of the sample without the addition of cell lysate (see FIG. 4A to 4C). Meanwhile, in strain 4, no high fluorescence was detected in any of the five pairs of probe sets used in the experiment (see FIG. 4D).

This confirms that strains 1, 2, and 3 are functional lactic acid bacteria belonging to Lactiplantibacillus sp., that are capable of biosynthesizing C30 carotenoids.

In addition, it was found that the difference in fluorescence values between the presence and absence of the target gene in the probes used in the experiment was statistically significant (ANOVA test, ****: p<0.0001, ***: p<0.001, **: p<0.01), which means that through simple pretreatment after isolation of lactic acid bacteria, it is easy to distinguish the presence or absence of useful functional microorganisms by the difference in signal intensity in the strains.

### Example 4. Verification of Usability as Detection Method for Rare Lactic Acid Bacteria

In the present Example, the results of the isothermal one-pot reaction performed in Example 3 above were compared with the conventional gene cluster detection method to confirm the accuracy of the rare cluster detection technology of lactic acid bacteria with the isothermal one-pot reaction technology.

### A. Detection of C30 Carotenoid Biosynthetic Essential Gene

Genome mining is a method of targeting DNA to detect biosynthetic gene clusters, and is the most basic method used to isolate functional microorganisms having useful activity, despite the human and time-consumption and the difficulties associated with the complexity of the steps, as the amplification of DNA genes allows accurate results to be obtained from the amplified DNA fragments (PCR amplicons). Therefore, PCR was additionally performed to reassess the reliability of the results of the isothermal one-pot reaction performed in Example 3.

PCR was performed by extracting the genomic DNA of the lactic acid bacteria to be analyzed, and primers were prepared, respectively, to detect the C30 carotenoid biosynthetic essential genes.

Table 4 below shows the primers used in this experiment. For Enterococcus gilvus (E. gilvus), regions matching the sequence of Enterococcus sp., at the 5′ and 3′ ends of the C30 carotenoid essential biosynthetic gene were prepared as primers and used.

TABLE 4

| | | Sequence Information | Amplification |
|---|---|---|---|
| Primer type | Type | (5′→-3′ ) | Size |
| Lactiplanti- | Forward | SEQ ID NO: 25 | 2,379 bp |
| bacilus | | ATG AAG CAA GTA TCG ATT ATT | |
| sp. | | GGC G | |
| | Reverse | SEQ ID NO: 26 | |
| | | TTA AGC CTC CTT AAG GGC TAG | |
| Enterococcus sp. | Forward | SEQ ID NO: 27 | 2,377 bp |
| | | ATG AAG AAG GTA ATA GTA ATA | |
| | | GGT G | |
| | Reverse | SEQ ID NO: 28 | |
| | | TCG CGG TAA ACA AAA GTT GG | |
| L. citreum | Forward | SEQ ID NO: 29 | 2,373 bp |
| | | ATG CAG AAC ATT TCA ATA GTA | |
| | | GGC | |
| | Reverse | SEQ ID NO: 30 | |
| | | TTA TGC CAC AAT ATA TTT CAC | |
| | | CGC | |
| L. mesenteroides | Forward | SEQ ID NO: 31 | 2,364 bp |
| | | ATG AAA ACA ATC TCA ATA ATT | |
| | | GGA G | |
| | Reverse | SEQ ID NO: 32 | |
| | | TCA CTT AAT GTG TTT TGA AAA | |
| | | GG | |

To proceed with the identification of the strain along with the detection of the C30 carotenoid biosynthetic essential gene from the gDNA extracted from the strain, PCR was performed with the compositions in Table 5 below.

TABLE 5

| Reagent | Concentration | Amount | |
|---|---|---|---|
| Genomic DNA | | 1 | [symbol] |
| Forward primer | 10 pmole | 2.5 | [symbol] |
| Reverse primer | 10 pmole | 2.5 | [symbol] |
| Q5 polymerase | | 0.5 | [symbol] |
| Q5 reaction buffer | 5X | 10 | [symbol] |
| dNTP | 2.5 mM | 4 | [symbol] |
| Nuclease free water | | 29.5 | [symbol] |
| Total | | 50 | [symbol] |

PCR was performed at 98° C. for 30 seconds, followed by 35 cycles of 10 seconds at 98° C., 30 seconds at the annealing temperature of each primer, and 2 minutes at 72° C. Subsequently, a final extension step was performed at 72° C. for 3 minutes, and then the temperature was lowered to 4° C. The PCR amplicon was then purified, and the position of the bands was confirmed by agarose gel electrophoresis to determine whether DNA was amplified. The gel used was a 1% agarose gel, which was run at 135V for 25 minutes.

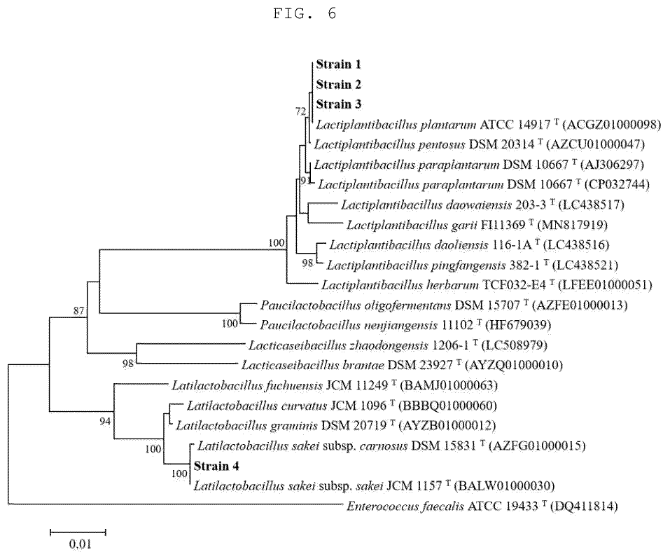

As shown in FIG. 5, in strains 1, 2, and 3, electrophoretic bands were only confirmed in PCR amplicons from PCR performed using primers capable of detecting the C30 carotenoid essential biosynthetic gene of Lactiplantibacillus sp. In contrast, no electrophoretic band could be confirmed in Strain 4. This confirms that lactic acid bacteria that are predicted to have the C30 carotenoid gene cluster must be subjected to gene detection, even if they are subjected to media testing and screening by colony color.

Furthermore, this experiment verified that the results of the isothermal one-pot reaction and the genomic mining results were consistent, thus confirming that the detection of C30 carotenoid essential biosynthetic genes by isothermal one-pot reaction can be effectively achieved.

### B. Identification of Lactic Acid Bacteria Targeted in the Reaction

Unlike the isothermal one-pot reaction, when genome mining techniques are used to detect useful lactic acid bacteria, strain identification must be performed separately, and thus universal primers were used, respectively, as shown in Table 6 below.

TABLE 6

| Primer | | Sequence Information | Amplification |
|---|---|---|---|
| type | Type | (5′→-3′) | Size |
| 16S rRNA | Forward | SEQ ID NO: 33 | About 1.5 kb |
| | | AGA GTT TGA TCM TGG CTC AG | |
| | Reverse | SEQ ID NO: 34 | |
| | | GGT TAC CTT GTT ACG ACT TC | |

Information about the reagents used in the PCR is shown in Table 5 above. PCR was performed at 98° C. for 30 seconds, followed by 35 cycles of 10 seconds at 98° C., 30 seconds at the annealing temperature of each primer, and 1 minute 30 seconds at 72° C. Subsequently, a final extension step was performed at 72° C. for 3 minutes, and then the temperature was lowered to 4° C. The PCR amplicon was then purified, and the position of the bands was confirmed by agarose gel electrophoresis to determine whether DNA was amplified. The gel used was a 1% agarose gel, which was run at 135V for 25 minutes.

As shown in FIG. 6, strains 1, 2, and 3 were identified as Lactiplastibacillus plantarum (L. plantarum). Since Lactiplantibacillus plantarum has been reported as a strain with a C30 carotenoid essential biosynthetic gene, conserved region was considered when constructing DNA probes for detecting the C30 carotenoid cluster of the Lactiplantibacillus sp., and as a result, an identification consistent with an isothermal one-pot reaction was confirmed.

Strain 4, on the other hand, was identified as Lactilactobacillus sakei. Lactilactobacillus sakei has not reported to produce C30 carotenoids, and the conserved region was not considered even when constructing the DNA probe for detecting the C30 carotenoid cluster of lactic acid bacteria, which is why no fluorescence value was confirmed in the isothermal one-pot reaction.

These results are consistent with the results of the isothermal one-pot reaction in Example 3, confirming that the detection of C30 carotenoid-biosynthetic functional lactic acid bacteria can be readily achieved from cell lysates of the strain with only a simple pretreatment.

Therefore, the isothermal one-pot reaction-based isolation of C30 carotenoid-biosynthetic lactic acid bacteria enables high-speed detection with simplified procedures, and provides two pieces of information in one reaction, including the isolation of C30 carotenoid biosynthetic functional lactic acid bacteria and the identification of the strain, which can be used for rapid and accurate isolation of functional microorganisms.

The platform according to the present disclosure selectively binds only to the functional metabolite biosynthetic gene cluster by using the probe set according to the present disclosure, and thus it is possible to perform phenotype-based screening on strains isolated using the probe set according to the present disclosure, thereby effectively selecting strains with biologically active biosynthetic pathway (functional metabolite biosynthetic strains) in a fast and accurate manner. Furthermore, the platform according to the present disclosure is able to utilize RNA as well as DNA as targets, thereby detecting gene clusters that are actually actively expressed.

While the foregoing has described in detail certain aspects of the present disclosure, it will be apparent to one of ordinary skill in the art that these specific descriptions are merely preferred embodiments and are not intended to limit the scope of the disclosure. Therefore, the substantive scope of the disclosure will be defined by the appended claims and equivalents thereof.

What is claimed is:

1. A method for detecting functional metabolite biosynthetic strains, comprising:
(a) treating a sample with an isothermal one-pot reaction probe set for detecting a functional metabolite biosynthetic strain including a first probe and a second probe; and hybridizing the probe set to a target nucleic acid sequence;
(b) treating the hybridization product of step (a) above with a ligation agent to ligate the first and second probes of the probe set, and treating the thus-obtained ligation product with a polymerase to initiate transcription; and
(c) treating the transcription product of step (b) with an aptamer-reaction material to detect signal generation of an aptamer in the transcription product,
wherein the first probe is a promoter probe (PP) having a structure of the following general formula (I);
3′-X-Y-5′ (I)
in the general formula (I) above,
X is an upstream hybridization sequence (UHS) portion having a hybridization sequence complementary to the target nucleic acid sequence; the target nucleic acid sequence is DNA or RNA; Y is a stem-loop structure portion containing a promoter sequence recognizable by an RNA polymerase; and X and Y are deoxyribonucleotides; and
the second probe is a reporter probe (RP) having a structure of the following general formula II;
3′-Z-X′-5′ (II)
in the general formula (II) above,
Z is an aptamer sequence portion with an interactive labeling system comprising one label or a plurality of labels generating a detectable signal; the target nucleic acid sequence is DNA or RNA; X′ is a downstream hybridization sequence (DHS) portion having a hybridization sequence complementary to the target nucleic acid sequence; and Z and X′ are deoxyribonucleotides.

2. The method of claim 1, wherein the functional metabolite is at least any one selected from the group consisting of carotenoid, 1-deoxynojirimycin (DNJ), violacein, staurosporine, and rebeccamycin.

3. The method of claim 1, wherein the target RNA sequence is DNA or RNA of at least any one gene selected from the group consisting of gabT1, yktc1, gutB1, vioA, vioB, vioE, vioD, vioC, staP, staG, staN, staMA, staMB, rebH, rebo, rebD, rebC, rebP, rebG, rebM, crtB, crtI, lyeJ, cruF, crtEb, cruY, crtM, crtN, crtP, crtQ, and crtO.

4. The method of claim 1, wherein the probe set is a probe set for detecting any one strain selected from the group consisting of the genus Lactiplantibacillus, the genus Enterococcus, the genus Leuconostoc, Staphylococcus aureus, the genus Rhodococcus, the genus Gordonia, the genus Arthrobacter, the genus Haloferax, the genus Bacillus, Chromobacterium violaceum, Streptomyces staurosporeus, and Lentzea aerocolonigenes.

5. The method of claim 2, wherein the carotenoid is any one selected from the group consisting of C30 carotenoid, C40 carotenoid, and C50 carotenoid.

6. The method of claim 3, wherein the target nucleic acid sequence is DNA or RNA of at least any one gene selected from the group consisting of crtM, crtN, crtP, crtQ, crtO, crtB, crtI, lyeJ, cruF, crtEb, and cruY.

7. The method of claim 4, wherein the genus Lactiplantibacillus comprises Lactiplantibacillus plantarum.

8. The method of claim 4, wherein the genus Enterococcus comprises at least any one selected from the group consisting of Enterococcus casseliflavus, Enterococcus faecalis, Enterococcus faecium, Enterococcus gallinarum, Enterococcus mundtii, and Enterococcus saccharolyticus.

9. The method of claim 4, wherein the genus Leuconostoc comprises Leuconostoc citreum.

10. The method of claim 1, wherein a functional metabolite is 1-deoxynojirimycin (DNJ), and a target nucleic acid sequence is DNA or RNA of at least any one gene selected from the group consisting of gabT1, yktc1, and gutB1.

11. The method of claim 10, wherein the probe set is a probe set for detecting strains of the genus Bacillus.

12. The method of claim 11, wherein the strain of the genus Bacillus is any one selected from the group consisting of Bacillus subtilis, Bacillus atrophaeus, Bacillus velezensis, and Bacillus amyloliquefaciens.

13. The method of claim 1, wherein a functional metabolite is violacein, and a target nucleic acid sequence is DNA or RNA of at least any one gene selected from the group consisting of vioA, vioB, vioE, vioD and vioC.

14. The method of claim 13, wherein the probe set is a probe set for detecting strains of Chromobacterium violaceum.

15. The method of claim 1, wherein a functional metabolite is staurosporine, and a target nucleic acid sequence is DNA or RNA of at least any one gene selected from the group consisting of staP, staG, staN, staMA, and staMB.

16. The method of claim 15, wherein the probe set is a probe set for detecting strains of Streptomyces staurosporeus.

17. The method of claim 1, wherein a functional metabolite is rebeccamycin, and a target nucleic acid sequence is DNA or RNA of at least any one gene selected from the group consisting of rebH, rebo, rebD, rebC, rebP, rebG, and rebM.

18. The method of claim 17, wherein the probe set is a probe set for detecting strains of Lentzea aerocolonigenes.

19. The method of claim 1, wherein the isothermal one-pot reaction is performed simultaneously in one vessel, without a separate amplification reaction, unified by any one specified temperature in the range of 15° C. to 50° C.

20. The method of claim 1, wherein the isothermal one-pot reaction is performed simultaneously, unified with a one-pot reaction buffer containing Tris-HCl, MgCl₂, NTPs, and extreme thermostable single-stranded DNA binding protein (ET-SSB).
